(19) United States
(12) Patent Application Publication
(10) Pub. No.: US 2024/0064500 A1
(43) Pub. Date: Feb. 22, 2024

(54) Transmission Unit for Bluetooth Pairing

(71) Applicant: Cosinuss GmbH, Munich (DE)

(72) Inventor: Johannes Kreuzer, Munich (DE)

(21) Appl. No.: 18/259,336

(22) PCT Filed: Dec. 24, 2021
(86) PCT No.: PCT/EP2021/084282

§ 371 Date: Jun. 26, 2023

(30) Foreign Application Priority Data
Dec. 24, 2020 (DE) 10 2020 216 572.6

(51) Int. Cl. H04W 4/80

(52) U.S. Cl. CPC H04W 4/80

(57) ABSTRACT

The present invention relates to a transmission unit for a signal out of the bandwidth in a Bluetooth pairing of at least two Bluetooth-enabled devices, a device for measuring vital parameters, a system and a method for Bluetooth pairing.

The present invention relates to a transmission unit for a signal outside the bandwidth in a Bluetooth pairing of at least two Bluetooth-enabled devices, a device for measuring vital parameters, a system and a method for Bluetooth pairing.

### BACKGROUND ART TECHNOLOGY

Progressive miniaturization allows the measurement of vital parameters even during physical movement, ideally using sensors in the ear canal. These have the advantage that, unlike wrist sensors, they provide stable and continuous readings. Wrist sensors, on the other hand, can be subject to strong interference, especially during sports, which can falsify the measured values. Advantageously, the ear canal sensors can be wirelessly connected via Bluetooth to other Bluetooth-enabled devices, in particular to mobile phones, via a Bluetooth interface.

Continuous measurements of vital parameters in the external auditory canal are carried out in the fields of individual sport activities, but are also increasingly being used in the medical sector. The device used therefor comprises a light emitter, usually in the form of an LED emitting in the green, red or infrared ranges, with the light emitter being placed in the external auditory canal on the skin of the auditory canal using an elastic silicone shield. At a remote point, the light emerging from the tissue is detected with a photodiode and converted into electricity, which is detected in a measuring unit arranged behind the auricle. This measuring unit houses measuring technology, power supply of the device and a processor. Analogue signals are digitized, processed and sent to appropriate receiver devices via Bluetooth, ANT+ or other wireless protocols.

For the transmission of data via Bluetooth, in particular for the transmission of individually measured and assigned vital parameters, the problem arises that, on the one hand, due to the type of information to be transmitted in the medical field and due to data protection regulations, on the other hand a secure data transmission and storage is absolutely necessary, but on the other hand the devices for measuring vital parameters must be kept small, compact and user-friendly. Devices that have a user interface for entering PIN codes or the like for Bluetooth pairing are generally ruled out as devices for measuring vital parameters, because they are too large and susceptible to interference.

The European patent application No. EP 2 717 756 A2 discloses a sensor for measuring a vital parameter in a person's auditory canal. However, the sensor only provides for the possibility of a secure data transmission to a Bluetooth-enabled device if a pairing has been carried out beforehand. However, the pairing is the point of attack here.

### Disclosure of the Invention, Problem, Solution, Advantages

Starting from the above considerations, the present invention is therefore based on the problem of providing transmission units for a signal outside the bandwidth in a Bluetooth pairing of at least two Bluetooth-enabled devices, a device for measuring vital parameters, a system and a method for Bluetooth pairing, wherein the manual entry of authentication codes or for unsecured transmission can be dispensed with and the problems described above can thus be avoided.

In a first aspect, the present disclosure therefore relates to a transmission unit for a signal outside the bandwidth in a Bluetooth pairing of at least two Bluetooth-enabled devices. The transmission unit can be provided with at least one shielded receptacle for a Bluetooth-enabled device. The receptacle can be provided with a signal source and/or with a signal detector. A Bluetooth-enabled device positioned in the receptacle can receive and/or transmit signals in a shielded manner, i.e., shielded. The signals can represent a Bluetooth pairing code.

In a preferred implementation, the signal can also represent device-specific or patient-specific data, such as a configuration or a patient identification number (patient ID), in addition to or where appropriate as an alternative to the Bluetooth pairing code.

According to the invention, a signal outside the bandwidth in a Bluetooth pairing of at least two Bluetooth-enabled devices is understood as meaning any signal whose frequency is outside the frequency bandwidth, which is typically used in the context of a Bluetooth pairing (out-of-band).

According to the invention, a shielded receptable means any receptable or recess, which mechanically or by other means can shield the signal transmission between the Bluetooth-enabled device and the signal source or the signal detector from the environment of the transmission unit. In other words, unwanted signal reception by third-party devices outside the system transmission unit—Bluetooth-enabled device is prevented when the device is positioned in the shielded receptacle.

According to the invention, a Bluetooth pairing code is understood to be any pairing code for a Bluetooth connection that can provide a secure connection between two Bluetooth-enabled devices. For example, the Bluetooth pairing code can be a four-digit pairing code, or a six-digit pairing code, or an eight-digit pairing code. Typically, the Bluetooth pairing code is a four-digit alphanumeric code.

Through out-of-band management outside of the Bluetooth frequency range, a network operator can define trust limits when accessing network components. Out-of-band management can also be used to ensure connectivity (including the ability to determine the status of one network component) independent of the status of other in-band network components.

In a second aspect: the present invention relates to a device for measuring vital parameters. The device can have a Bluetooth module for pairing the device with another Bluetooth-enabled device for a wireless data transmission. The device for measuring vital parameters can also have a processor connected to the Bluetooth module. The vital parameters measurement device may further include a signal detector coupled to the processor. The device for measuring vital parameters can also include storage means connected to the processor. The storage means may include instructions for the processor to receive a Bluetooth pairing code and/or device specific or patient specific data representing a received signal.

According to the invention, a device for measuring vital parameters is understood to be any device that can be used to measure vital parameters on a human or on an animal. According to the invention, the Bluetooth-enabled device according to the first aspect of the invention can be used as the device for measuring vital parameters according to the second aspect of the invention.

According to the invention, any device that can detect an information-carrying signal is used as a signal detector. However, the signal detector is preferably a light detector. If the signal detector is a light detector, the signal transmission can be achieved particularly reliably and securely.

According to the invention, a signal is understood as meaning any signal that can carry information. The signal is preferably a light signal with a defined amplitude and frequency. The light signal can be either a monochromatic light signal or a polychromatic light signal. The polychromatic signal can have a specific LED color spectrum suitable for the Bluetooth-enabled device. Ideally, the signal is discrete in terms of time and value. For example, the signal can encode a Bluetooth pairing code. Additionally, or alternatively, the signal can also encode parameters for the configuration and/or a patient ID.

The device for measuring vital parameters can be configured such that the signal detector receives a signal, e.g., a defined light signal, which represents a Bluetooth pairing code and/or device-specific or patient-specific data. The Bluetooth pairing code can be sent to another device. Advantageously, the signal detector connected to the processor, for example a photodiode, can be used both for measuring vital parameters and for receiving signals representing a Bluetooth pairing code.

In a preferred implementation, the device for measuring vital parameters additionally has a signal source connected to the processor for sending out a signal. The storage means may preferably include instructions for the processor to emit a signal representing a Bluetooth pairing code and/or device specific or patient specific data.

The device for measuring vital parameters can also be configured so that a signal source, e.g., a light source, can emit a signal, e.g., a defined light signal representing a Bluetooth pairing code and/or device-specific or patient-specific data. This pairing code can be used to provide a Bluetooth pairing code, which is a representation of the transmitted signal, in another device that is designed with a corresponding signal detector. In this case, the device can be configured as a master. The storage means are preferably designed to store at least one Bluetooth pairing code and/or device-specific or patient-specific data. In a preferred implementation, the storage means can be capable of storing a large number of Bluetooth pairing codes and/or device-specific or patient-specific data, which can be logically linked to time-specific and device-specific information.

Preferably, the signal detector is a photodetector (i.e., a light detector). Any electronic component which can convert light into an electrical signal using a photoelectric effect or which exhibits an electrical resistance dependent on incident light radiation can be used as a photodetector. According to the invention, the signal detector can be selected from the group consisting of photocells, photomultipliers, microchannel plate photomultipliers, CMOS sensors, CCD sensors, photodiodes, phototransistors and photoresistors. Ideally, the signal detector is an inorganic or organic photodiode or phototransistor.

Preferably the signal source is a light source. Any electronic component that can emit light with a defined wavelength distribution according to frequency and intensity can be used as the light source. According to the invention, the signal source is preferably a light-emitting diode, i.e., a light-emitting diode.

The device is preferably capable of measuring vital parameters in the auditory canal.

In a third aspect, the invention relates to a system comprising a device according to the invention for measuring vital parameters and a transmission unit. The receptable can be provided with a signal source and/or with a signal detector. A device for measuring vital parameters positioned in the receptable can receive shielded signals and/or emit data representing a Bluetooth pairing code and/or device-specific or patient-specific data.

The shielding of the signal transmission can preferably be achieved in that the device and the receptacle of the transmission unit are opaque and when the device is positioned in the receptacle of the transmission unit, the signal receiver and the signal detector are positioned in close proximity inside the system. In this case, the transmission unit and the device itself can serve as a shield. The device can preferably be positioned in a form-fitting manner in the receptacle of the transmission unit, resulting in very good shielding. Alternatively, the receptacle of the transmission unit can also be a concave, translucent recess or a tubular, opaque recess into which a convex or elongated Bluetooth module can be inserted or inserted. In this case, the opaque recess and the spatial closure of the inserted or pushed-in Bluetooth module provide the shielding.

The transmission unit and/or the device preferably also has mechanical or magnetic positioning or locking aids. With the aid of the positioning or locking aids, the signal receiver and the signal detector can be positioned or locked in spatial proximity, so that transmission is as efficient as possible. For example, the device with magnets as positioning aids can be detachably connected to the transmission unit, whereby the signal receiver and the signal detector can be brought into spatial proximity in a shielded manner. Alternatively, the device with mechanical holding means. e.g., Velcro connections or by friction, as positioning aids are releasably connected to the transmission unit, the signal receiver and the signal detector can be brought in spatial proximity in a shielded manner.

In addition to the actual device for measuring vital parameters, the system can thus comprise a transmission unit which is provided with a signal shielded receptable. This receptable can contain a signal source or a signal detector or a combination of both, so that a device positioned in the recording can both, receive a Bluetooth pairing code via a corresponding signal (slave) and generate a Bluetooth pairing code via a corresponding signal (master).

The transmission unit can be integrated into another Bluetooth-enabled device; it is preferably present as a separate transmission unit.

The transmission unit is preferably capable of transmitting a defined signal, e.g., a defined light signal, for providing a Bluetooth pairing code in the device.

The transmission unit is preferably capable of transmitting a defined signal, e.g., a defined light signal, to provide a Bluetooth pairing code.

In a fourth aspect, the present invention relates to a method for connecting the device for measuring vital parameters according to the invention to another Bluetooth-enabled device. The method can include the following steps:

Step a): Receiving a defined signal by the device for measuring vital parameters, wherein the defined signal is transmitted from a signal source to the signal detector of the device for measuring vital parameters, wherein the signal source and the signal detector are shielded.

Step b): Evaluating the defined signal as a Bluetooth pairing code.

Step c): Using the Bluetooth pairing code to establish a Bluetooth connection between the device for measuring vital parameters and the other Bluetooth-enabled device.

Preferably, the method of connecting the device for measuring vital parameters with another Bluetooth-enabled device includes the following step a′).

The step a′) includes the transmission of a defined signal by the device for measuring vital parameters, wherein the defined signal is transmitted from a signal source of the device to a signal detector, wherein the signal source and the signal detector are shielded.

Preferably, the method according to the fourth aspect of the present invention uses the system according to the third aspect of the present invention.

### PREFERRED EMBODIMENT OF THE INVENTION

Some particular embodiments of the invention are described below by way of example and not in an exhaustive manner. The specific embodiments only serve to explain the general inventive idea, but they do not limit the invention.

In one possible embodiment, the transmission unit has a receptable for a Bluetooth-enabled device, with the receptable being provided with an LED light source. A device for measuring vital parameters in a possible embodiment has a Bluetooth module for pairing the device with another Bluetooth-enabled device for wireless data transmission, a processor connected to the Bluetooth module, an inorganic photodiode connected to the processor as a signal detector and with storage means connected to the processor and containing instructions for the processor. The instructions serve to receive a Bluetooth pairing code and/or device-specific or patient-specific data that represent a received signal. The storage means also includes instructions for the processor to emit a signal representing a Bluetooth pairing code and/or device specific or patient specific data. The storage means can store the Bluetooth pairing code to be received or to be sent and/or device-specific or patient-specific data. The device for measuring vital parameters is shaped and sized to measure vital parameters in the ear canal.

In order to transmit the Bluetooth pairing code and/or device-specific or patient-specific data, the device with the Bluetooth module can be placed or plugged into the shielded receptacle of the transmission unit. This enables a secure, non-interceptable transmission of the Bluetooth pairing code and/or device-specific or patient-specific data from the LED light source to the inorganic photodiode. External third parties have no way of intercepting the signal optically or in any other way during the transmission process. The signal can be transmitted by changing the light intensity, the transmission frequency, or a combination thereof. As a sensor, the photodiode recognizes the Bluetooth pairing code and uses this as the encryption code for all subsequent Bluetooth connections.

The combination of the transmitter and the device for measuring vital parameters can be used in a system to receive and/or to transmit shielded signals representing a Bluetooth pairing code and/or device-specific or patient-specific data, or can also be used in a method in which the device is connected to a mobile phone as another Bluetooth-enabled device.

1. A transmission unit for a signal outside the bandwidth in a Bluetooth pairing of at least two Bluetooth-enabled devices, wherein the transmission unit is provided with at least one shielded receptacle for a Bluetooth-enabled device, wherein the receptacle is provided with a signal source and/or with a signal detector such that a Bluetooth-enabled device positioned in the receptacle can receive and/or transmit in a shielded manner signals which represent a Bluetooth pairing code and/or device-specific or patient-specific data, such as a configuration or a patient identification number.

2. A device for measuring vital parameters, comprising:
a Bluetooth module for pairing the device with another Bluetooth-enabled device for wireless data transmission, a processor connected to the Bluetooth module; a signal detector connected to the processor; and storage means coupled to the processor and comprising instructions for the processor to receive a Bluetooth pairing code and/or device specific or patient specific data representing a received signal.

3. The device according to claim 2, further comprising a signal source connected to the processor for emitting a signal, wherein the storage means comprises instructions for the processor to emit a signal representing a Bluetooth pairing code and/or device specific or patient specific data.

4. The device according to any one of claim 2 or 3, wherein the storage means are capable of storing at least one Bluetooth pairing code and/or device-specific or patient-specific data.

5. The device according to any one of the preceding claims, wherein the signal detector is provided in form of an inorganic or organic photodiode or a phototransistor and/or wherein the signal source is provided in form of a light-emitting diode.

6. The device according to any one of the preceding claims, wherein the device is capable of measuring vital parameters in the auditory canal.

7. A system comprising a device for measuring vital parameters according to any one of claims 2 to 6,
a transmission unit according to claim 1, wherein the receptable is provided with a signal source and/or with a signal detector, such that a device for measuring vital parameters positioned in the receptable can receive and/or emit shielded signals representing a Bluetooth pairing code and/or device-specific or patient-specific data.

8. The system according to claim 7, wherein the transmission unit is capable of emitting a defined signal in order to provide a Bluetooth pairing code and/or device-specific or patient-specific data in the device and/or is capable of receiving a defined signal in order to provide a Bluetooth pairing code and/or device-specific or patient-specific data.

9. A method for connecting a device for measuring vital parameters according to any one of claims 2 to 6 with another Bluetooth-enabled device, comprising:
a) receiving a defined signal by the device for measuring vital parameters, wherein the defined signal is transmitted from a signal source to the signal detector of the device for measuring vital parameters, the signal source and the signal detector being shielded,
b) evaluating the defined signal as a Bluetooth pairing code,
c) using the Bluetooth pairing code to establish a Bluetooth connection between the device for measuring vital parameters and the other Bluetooth-enabled device.

10. The method according to claim 9 additionally comprising the step of:
a′) transmission of a defined signal by the device for measuring vital parameters, wherein the defined signal is transmitted from a signal source of the device to a signal detector, wherein the signal source and the signal detector are shielded.

11. A method according to any one of claim 9 or 10, using the system according to any one of claim 7 or 8.
